SEQUENCE LISTING (8 sequences)

SEQ ID NO: 1 - DNA, 438 bp, Homo sapiens
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtatgtgacaagccgaggcggtga

SEQ ID NO: 2 - DNA, 438 bp, Homo sapiens
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatagcgagatgagagagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtatgtgacaagccgaggcggtga

SEQ ID NO: 3 - protein, 145 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 4 - protein, 145 aa, Homo sapiens
APMAEGGGQNHHEVVKFMDVYQRSYCHPIETLVDIFQEYPDEIEYIFKPSCVPLMRCGGCCNSEMRECVPTEESNITMQIMRIKPHQGQHIGEMSFLQHNKCECRPKKDRARQEKKSVRGKGKGQKRKRKKSRYKSWSVCDKPRR

SEQ ID NO: 5 - DNA, 27 bp, Artificial sequence
  Single strand DNA oligonucleotide
cgggatccgaaaccatgaactttctgc

SEQ ID NO: 6 - DNA, 46 bp, Artificial sequence
  Single strand DNA oligonucleotide
tcctcagtgggcacacactctctcatctcgctattgcagcagcccc

SEQ ID NO: 7 - DNA, 31 bp, Artificial sequence
  Single strand DNA oligonucleotide
gagtgtgtgcccactgaggagtccaacatca

SEQ ID NO: 8 - DNA, 28 bp, Artificial sequence
  Single strand DNA oligonucleotide
ggggtacccctcaccgcctcggcttgtc

(19) United States
(12) Patent Application Publication
(10) Pub. No.: US 2006/0252919 A1
(43) Pub. Date: Nov. 9, 2006

(54) Vegf variant that lacks vegfr-1 binding activity and its use in promotion of re-endothelization and prevention of in-stent restenosis

(75) Inventor: Gera Neufeld, HAIFA (IL)

Correspondence Address: c/o Anthony Castorina, Suite 207, 2001 Jefferson Davis Highway, Arlington, VA, 22202, US

(73) Assignee: TECHNION RESEARCH & DEVELOPMENT FOUNDATION LTD., HAIFA (IL)

(21) Appl. No.: 10/539,289

(22) PCT Filed: Dec. 17, 2003
(86) PCT No.: PCT/IL03/01085

§ 371 Date: Jun. 16, 2005

Related U.S. Application Data
(60) Provisional application No. 60/433,778, filed on Dec. 17, 2002.

(51) Int. Cl. C07K 14/475; C12Q 1/68; C07H 21/04; C12P 21/06

(52) U.S. Cl. 530/399; 435/6; 435/69.1; 435/320.1; 435/325; 536/23.5

(57) ABSTRACT

A VEGF₁₄₅ polypeptide devoid of a VEGFR-1 binding activity and methods of making and using same in preventing and/or treating restenosis are provided.

### FIELD AND BACKGROUND OF THE INVENTION

The present invention relates to a VEGF variant and to methods of producing and using same in preventing and treating restenosis.

Percutaneous coronary intervention (PCI) which involves the placement of coronary stents represents an attractive alternative to surgical revascularization. However, due to the process of de-endothelialization, i.e., the removal of endothelial cells by the stent itself, in-stent restenosis occur in 15-50% of the cases within 6 to 9 months following intervention. Immediately following stent placement, a layer of activated platelets and fibrin is deposited on the denuded surface. The activated platelets stimulate the attachment of circulating leukocytes to the site of the stent and initiate leukocytes rolling along the injured surface. At the same time, the release of cytokines and growth factors from smooth muscle cells (SMCs), platelets and resident leukocytes stimulates the binding of leukocytes to the layer of platelets and fibrin as well as the proliferation and migration of SMCs to the neointima that covers the denuded surface. With time, the neointima consists of fewer cellular elements and more ECM components. In parallel, following stent placement a process of re-endothelialization begins. Growth factors that accelerate re-endothelialization include factors belonging to the VEGF family [Welt, F. G. and Rogers, C., (2002). Inflammation and restenosis in the stent era. Arterioscler. Thromb. Vasc. Biol. 22: 1769-1776]. Re-endothelialization in this case is the result of the interaction of VEGF with the VEGFR-2 receptor on the endothelial cells. This interaction is accompanied by the generation of nitrogen mono oxide (NO) by the endothelial cells, leading to inhibition of SMC migration and proliferation [Rutanen, J., Puhakka, H., and Yla-Herttuala, S. (2002) Post-intervention vessel remodeling. Gene Ther. 9, 1487-1491].

One severe limitation of stent deployment is restenosis which is a process of re-narrowing or blockage of the artery as a result of ECM deposition and SMC migration at the site where an angioplasty or stent procedure has been taken place.

Several methods attempting to prevent restenosis have been suggested. These include coated stent systems and the stent eluting systems. For example, statins, the lipid lowering agents, were used in stent coatings. However, although statins are capable of upregulating the endothelial cell nitric oxide synthase level and nitric oxide synthesis [Hemandez-Presa, et al. 2002. Atorvastatin reduces the expression of cyclooxygenase-2 in a rabbit model of atherosclerosis and in cultured vascular smooth muscle cells. Atheroslerosis, 160: 4958] in vivo experiments using atrovastatins resulted in no significant effect on the proliferation of the neointima although the degree of inflammation was reduced [Scheller B, et al., 2003. Atorvastatin stent coating does not reduce neointimal proliferation after coronary stenting. Z Kardiol. 92(12): 1025-8]. In addition, the dexamethasone-eluting stents [Liu X, et al., 2003. Study of antirestenosis with the BiodivYsio dexamethasone-eluting stent (STRIDE): a first-in-human multicenter pilot trial. Catheter Cardiovasc Interv. 60: 172-9], the sirolimus-eluting stents (Lemos Pa., et al., 2003. Coronary restenosis after sirolimus-eluting stent implantation: morphological description and mechanistic analysis from a consecutive series of cases. Circulation. 108: 257-60) and the paclitaxel-eluting stents (Tanabe K, et al., 2003. TAXUS III Trial: in-stent restenosis treated with stent-based delivery of paclitaxel incorporated in a slow-release polymer formulation. Circulation. 107: 559-64) have yet to be proven effective in preventing restenosis.

Another approach to prevent restenosis utilizes vascular brachytherapy, in which sealed sources of radioactive material deliver radiation at a very short distance to inhibit the proliferation of smooth muscle cells (neointima formation) which causes restenosis (Reisman M and Gray W A. J. 2003. Vascular brachytherapy and the strontium 90 vascular brachytherapy system. Invasive Cardiol. 15: 520-2). However, the efficacy of brachytherapy in treating restenosis is limited to only 30-66% of the cases.

Thus, the overall success of stent placement depends on the balance between re-endothelialization on one hand and the process of arterial sclerosis at the site of injury on the other hand [Welt, F. G. and Rogers, C. (2002) Inflammation and restenosis in the stent era. Arterioscler. Thromb. Vasc. Biol. 22: 1769-1776].

There is thus a widely recognized need for, and it would be highly advantageous to have, a method of accelerating re-endothelialization while at the same time inhibiting the migration and proliferation of SMCs thereby being useful in, for example, prevention and treatment of restenosis.

### SUMMARY OF THE INVENTION

According to one aspect of the present invention there is provided an isolated VEGF₁₄₅ polypeptide devoid of a VEGFR-1 binding activity.

According to another aspect of the present invention there is provided an isolated polynucleotide comprising a nucleic acid sequence encoding a VEGF₁₄₅ polypeptide devoid of a VEGFR-1 binding activity According to yet another aspect of the present invention there is provided a method of promoting re-endothelialization in a tissue of an individual comprising providing to the tissue of the individual a VEGF₁₄₅ polypeptide exhibiting a VEGFR-2 binding activity and lacking a VEGFR-1 binding activity thereby promoting re-endothelialization in the tissue of the individual

According to still another aspect of the present invention there is provided a method of preventing and/or treating restenosis in an individual in need thereof comprising providing to the individual a VEGF₁₄₅ polypeptide exhibiting a VEGFR-2 binding activity and lacking a VEGFR-1 binding activity thereby preventing and/or treating restenosis in the individual in need thereof.

According to an additional aspect of the present invention there is provided a nucleic acid construct comprising the isolated polynucleotide.

According to further features in preferred embodiments of the invention described below, the polypeptide is set forth by SEQ ID NO:4.

According to still further features in the described preferred embodiments the polypeptide is capable of binding to VEGFR-2.

According to still further features in the described preferred embodiments the polynucleotide is set forth by SEQ ID NO:2.

According to still further features in the described preferred embodiments the polynucleotide is at least 90% homologous to the polynucleotide sequence set forth by SEQ ID NO:1 as determined using the BlastN software of the National Center of Biotechnology Information (NCBI) using default parameters.

According to still further features in the described preferred embodiments the nucleic acid construct further comprising a promoter for directing expression of the isolated polynucleotide in mammalian cells.

According to still further features in the described preferred embodiments the promoter is an endothelial cell specific promoter.

According to still further features in the described preferred embodiments the tissue is selected from the group consisting of an artery and a vein.

According to still further features in the described preferred embodiments providing is effected by administering said VEGF₁₄₅ polypeptide into the tissue of the individual.

According to still further features in the described preferred embodiments providing is effected by expressing a polynucleotide encoding said VEGF₁₄₅ polypeptide in the tissue of the individual.

The present invention successfully addresses the shortcomings of the presently known configurations by providing a VEGF₁₄₅ polypeptide which lacks a VEGFR-1 binding activity and methods of using same in preventing and/or treating restenosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b illustrate the nucleotide sequence of the cDNAs encoding native (mature) and variant VEGF₁₄₅ proteins. FIG. 1a=native VEGF₁₄₅ cDNA (SEQ ID NO:1); FIG. 2b=variant VEGF₁₄₅ cDNA (SEQ ID NO:2). Nucleotides represent the coding sequence of the mature protein and do not include the nucleotides coding for the first 26 amino acids of the signal peptide. Note that the highlighted GAC, GGC and CTG codons in the cDNA encoding the native VEGF₁₄₅ (FIG. 1a) are replaced with the highlighted AGC, ATG and AGA codons in the cDNA encoding the variant VEGF₁₄₅ protein (FIG. 1b).

FIGS. 2a-b illustrate the amino acid sequence of native (mature) and variant VEGF₁₄₅. FIG. 2a=native VEGF₁₄₅ amino acid sequence (SEQ ID NO:3); FIG. 2b=variant VEGF₁₄₅ amino acid sequence (SEQ ID NO:4). Amino acids are numbered from the beginning of the mature VEGF protein and do not include the first 26 amino acids of the signal peptide. Note that the Asp-63, Gly-65 and Leu-66 amino acids in the native VEGF₁₄₅ protein (FIG. 2a, highlighted D, G and L letters, respectively) are replaced with Ser-63, Met-65 and Arg-66 in the variant VEGF₁₄₅ protein (FIG. 2b, highlighted S, M and R letters, respectively).

### DESCRIPTION OF THE PREFERRED EMBODIMENTS

The present invention is of a VEGF₁₄₅ polypeptide which lacks a VEGFR-1 binding activity and methods of using same in preventing and/or treating restenosis.

The principles and operation of the methods of producing the VEGF₁₄₅ polypeptide according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Percutaneous coronary interventions (PCI), including stent placement and angioplasty, represent attractive approaches for the treatment of coronary disorders. However, in 15-50% of the cases, restenosis occurs 6-9 months following PCI. Restenosis initiates with the accumulation of leukocytes on a layer of platelets and fibrin which further induce smooth muscle cells (SMCs) migration and proliferation and results with the deposition of a collagen matrix that builds the neointima which accumulates and eventually narrows down the artery.

Thus, in order to effectively prevent restenosis, one must inhibit the process of neointima formation on one hand, and promote the process of re-endothelialization on the other hand.

Since the process of neointima formation depends on the activation of the VEGFR-1 receptor on leukocytes and SMCs, lack of VEGFR-1 activation would in effect lead to inhibition of neointima formation. On the other hand, activation of the VEGFR-2 receptor of endothelial cells would induce their proliferation and migration and contribute to rapid re-endothelialization, which would, as a result, inhibit SMCs migration and proliferation.

Thus, an agent that would activate the VEGFR-2 receptor on one hand but would be incapable of activating the VEGFR-1 receptor on the other hand can be used to prevent neointima formation and restenosis.

An example of such an agent is the VEGF₁₆₅ variant in which the VEGFR-1 binding domain was mutated (Li, B. et al., 2000. Receptor-selective variants of human vascular endothelial growth factor generation and characterization, J. Biol. Chem. 275: 29823-29828). However, this VEGF₁₆₅ variant exhibits relatively week binding to the extracellular matrix and is therefore not expected to be retained at the denuded area for prolonged periods, thus severely limiting its effectiveness as an inducer of re-endothelialization.

While reducing the present invention to practice. the present inventors have constructed a polynucleotide encoding a VEGF₁₄₅ variant that is capable of binding to the VEGFR-2 receptor of endothelial cells but is incapable of binding to the VEGFR-1 of SMCs and leukocytes and is thus highly suitable for use in the prevention of restenosis.

Thus, according to one aspect of the present invention there is provided an isolated polynucleotide comprising a nucleic acid sequence encoding a VEGF₁₄₅ polypeptide devoid of a VEGFR-1 binding activity (also referred to herein as the VEGF₁₄₅ polypeptide variant of the present invention).

As used herein the term VEGF₁₄₅ polypeptide refers to the vascular endothelial growth factor isoform 145 (VEGF₁₄₅) protein such as the human VEGF₁₄₅ (GenBank Accession number: NP_{—}003367) which is set forth by SEQ ID NO:3. The VEGF₁₄₅ polypeptide can be encoded by a naturally occurring or synthetic polynucleotide. According to preferred embodiments the polynucleotide used by the present invention is at least 90%, at least 95%, more preferably, at least 98% homologous to the polynucleotide sequence set forth by SEQ ID NO:1 as determined using the BlastN software of the National Center of Biotechnology Information (NCBI) using default parameters.

Examples of naturally occuring VEGF₁₄₅ polynucleotide sequences include the human VEGF₁₄₅ polynucleotide sequence (GenBank Accession number: NM_{—}003376) or any of its homologues (e.g., the rat VEGF₁₄₅, GenBank Accession number: AF222779). Synthetic polynucleotide sequences which encode a polypeptide at least 75%, preferably at least 80%, more preferably at least 85%, most preferably 90%-100% homologous to SEQ ID NO:3 as determined by the BlastP software of the National Center of Biotechnology Information (NCBI) using default parameters can also be used by the present invention to generate the variant described hereinunder.

In order to produce a VEGF₁₄₅ polypeptide devoid of a VEGFR-1 binding activity (i.e. the present variant), the coding sequence of the VEGF₁₄₅ needs to be modified in such a way that the VEGFR-1 binding domain is inactivated. Such modifications can be accomplished via, for example, site directed mutagenesis.

Examples of site directed mutagenesis which inactivate the VEGFR-1 binding domain are described in Li, B. et al., 2000, J. Biol. Chem. 275: 29823-29828 for VEGF₁₆₅ and in Example 1 of the Examples section which follows for VEGF₁₄₅. It will be appreciated that such mutations are sequence specific, thus leaving the rest of the polynucleotide sequence unchanged.

Methods of introducing site directed mutations are known in the art and include, for example, PCR directed mutagenesis. Briefly, a PCR reaction is performed on a DNA template using primers which are selected from the DNA template but include modified deleted or added nucleotides in order to direct the PCR reaction to generate a mutated sequence. The PCR conditions are usually adjusted to amplify the template using the modified primers. Such adjustments include for example, increasing the concentration of the magnesium chloride ions and/or reducing the annealing temperature and the selection of suitable PCR conditions are within the capabilities of one skilled in the art.

As is shown in Example 1 of the Examples section which follows the present inventor has introduced the following PCR directed mutations: GAC→AGC, GGC→ATG, and CTG→AGA at nucleotide positions 187-189, 193-195 and 196-198, respectively, of the VEGF₁₄₅ cDNA (SEQ ID NO:1); such mutations resulted in replacement of Aspartic acid, Glycine and Leucine amino acids (at positions 63, 65 and 66 of the mature VEGF₁₄₅ protein SEQ ID NO:3) with Serine, Methionine and Arginine amino acids respectively. Such modifications to the polypeptide sequence resulted in inactivation of the VEGFR-1 binding domain.

It will be appreciated that alternative modifications which result in inactivation of the VEGFR-1 binding domain of the VEGF₁₄₅ polypeptide (amino acid coordinates 63-67 of SEQ ID NO:3) are also encompassed by the present invention.

Several approaches can be utilized to synthesize the VEGF₁₄₅ polypeptide variant of the present invention.

Methods of synthesizing recombinant proteins are well known in the art and include for example the use of in vitro transcription-translation systems and expression systems which enable expression and accumulation of the recombinant polypeptide in mammalian cells (e.g., HeLa cells, Cos cells), yeast cells (e.g., AH109, HHY10, KDY80), insect cells (e.g., Sf9) or bacteria cells (e.g., JM109, RP437, MM509, SW10).

Preferably, the VEGF₁₄₅ variant polypeptide of the present invention is synthesized by ligating a polynucleotide sequence encoding the VEGF₁₄₅ variant (e.g., the polynucleotide set forth by SEQ ID NO:2) into a mammalian, yeast or bacterial expression vector. Examples of such vectors include but are not limited to the pcDNA3.1, pBK-CMV and pCI vectors which are suitable for use in mammalian cells, the pGBKT7, pLGADH2-lacZ and pBGM18 vectors which are suitable for use in yeast cells and the pACK02scKan, pMLBAD, pMLS7 vectors which are suitable for use in bacterial cells.

Once produced in a host cell, the recombinant VEGF₁₄₅ protein is preferably purified from soluble cell extracts using for example, heparin-Sepharose affinity chromatography as described [Poltorak, Z., et al. (1997) VEGF₁₄₅: a secreted VEGF form that binds to extracellular matrix. J. Biol. Chem. 272, 7151-7158].

Purified VEGF₁₄₅ proteins can be further tested for their receptor binding specificity using methods known in the art, including for example, binding assays to porcine aortic endothelial (PAE) cells which express the recombinant VEGFR-1 and VEGFR-2 receptors. Briefly, increasing concentrations of ¹²⁵I-VEGF₁₄₅ polypeptide are added to the cells and are allowed to bind to the specific receptor. The degree of binding is correlated with the amount of radioactivity retains on the cells. Non-specific binding is determined using non-iodinated VEGF₁₄₅ essentially as described elsewhere [Gluzman-Poltorak, Z., et al. (2001) Vascular endothelial growth factor receptor-1 and neuropilin-2 form complexes. J. Biol. Chem. 276, 18688-18694; Soker, S., et al. (1998) Neuropilin-1 is expressed by endothelial and tumor cells as an isoform specific receptor for vascular endothelial growth factor. Cell 92, 735-745; Gluzman-Poltorak, Z., et al. (2000) Neuropilin-2 and Neuropilin-1 are receptors for 165-amino acid long form of vascular endothelial growth factor (VEGF) and of placenta growth factor-2, but only neuropilin-2 functions as a receptor for the 145 amino acid form of VEGF. J Biol Chem 275, 18040-18045].

As is mentioned hereinabove, VEGF₁₄₅ induces proliferation of endothelial cells upon binding to the VEGFR-2 receptor thereof. Such a mitogenic effect can be determined by employing a cell proliferation assay using human umbilical vein derived endothelial (HUVEC) cells and human aorta derived SMC as described (Poltorak, Z., et al., (1997) J. Biol. Chem. 272, 7151-7158; Gluzman-Poltorak, Z., et al. (2000). J Biol Chem 275, 18040-18045).

Moreover, the effect of the VEGF₁₄₅ polypeptide of the present invention on migration of SMC and HUVEC can be determined using scratch-wound assays [Savani, R. C., et al. (1995) Migration of bovine aortic smooth muscle cells after wounding injury. The role of hyaluronan and RHAMM. J. Clin. Invest 95, 1158-1168; Lemire, J. M., et al. (2002) Overexpression of the V3 variant of versican alters arterial smooth muscle cell adhesion, migration, and proliferation in vitro. J. Cell Physiol 190, 38-45; Itano, N., et al. (2002) Abnormal accumulation of hyaluronan matrix diminishes contact inhibition of cell growth and promotes cell migration. Proc. Natl. Acad. Sci. U.S. A 99, 3609-3614].

Since the VEGF₁₄₅ polypeptide of the present invention is capable of selectively activating the VEGFR-2 receptor on the endothelial cells it can be used to promote re-endothelialization of vascular tissue.

Thus, according to another aspect of the present invention there is provided a method of promoting re-endothelialization in vascular tissue of an individual.

As used herein the phrase “promoting re-endothelialization” refers to the proliferation and accumulation of endothelial cells upon activation of a VEGFR-2 receptor. Usually, re-endothelialization occurs following de-endothelialization, the process of removing endothelial cells from a tissue.

As used herein, the term “vascular tissue” refers to tissue of a blood vessel such as an artery or a vein.

According to preferred embodiments of the present invention the method is effected by providing to the tissue of the individual a VEGF₁₄₅ polypeptide exhibiting a VEGFR-2 binding activity and lacking a VEGFR-1 binding activity thereby promoting re-endothelialization in the tissue of the individual.

According to preferred embodiments of the present invention providing the VEGF₁₄₅ polypeptide to the tissue is effected by administering the VEGF₁₄₅ polypeptide into the tissue of the individual.

As used herein, “administering” refers to means for providing the VEGF₁₄₅ polypeptide into the tissue of the individual, using any suitable route, e.g., oral, sublingual intravenous, subcutaneous, percutaneous, transcutaneous, intramuscular, intracutaneous, intrathecal, epidural, intraoccular, intracranial, inhalation, rectal, vaginal, and the like administration.

Provision of the VEGF₁₄₅ polypeptide variant of the present invention can also be effected by administering to the individual an expressible polynucleotide encoding the VEGF₁₄₅ polypeptide variant.

Preferably, the polynucleotide encoding the VEGF₁₄₅ polypeptide variant of the present invention is administered as part of an expression vector which also includes a promoter suitable for directing expression of the VEGF₁₄₅ polypeptide variant in mammalian cells.

Constitutive promoters suitable for use with the present invention are promoter sequences which are active under most environmental conditions and most types of cells such as the cytomegalovirus (CMV) and Rous sarcoma virus (RSV). Tissue specific promoters suitable for use with the present invention include for example the endothelial specific promoter of Tie-2.

The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of VEGF₁₄₅ mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRepS, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

It will be appreciated that expression of the polynucleotide of the present invention using any of the methods described hereinabove results in the production of a VEGF₁₄₅ variant polypeptide lacking the VEGFR-1 binding activity but yet preserving the VEGFR-2 binding activity.

The VEGF₁₄₅ polypeptide variant of the present invention or the expression vector encoding same can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the VEGF₁₄₅ polypeptide variant or the expression vector encoding same which are accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (the VEGF₁₄₅ polypeptide or the expression vector encoding same) effective to prevent, alleviate or ameliorate symptoms of a disorder (e.g., restenosis) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to prevent restenosis (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The expression vector described above can be delivered into cells using a variety of delivery approaches, including, but not limited to, microinjection, electroporation, liposomes, epidermal patches, iontophoresis or receptor-mediated endocytosis. The selection of a particular method will depend, for example, on the cell into which the polynucleotide is to be introduced, as well as whether the cell is isolated in culture, or is in a tissue or organ in culture or in situ.

According to a preferred embodiment of the present invention, the expression vector encoding the VEGF₁₄₅ polypeptide variant of the present invention is administered into host tissues using a viral vector.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein.

Recombinant viral vectors are useful for in vivo expression of VEGF₁₄₅ since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

When retroviruses, for example, are used for polynucleotide transfer, replication competent retroviruses theoretically can develop due to recombination of retroviral vector and viral gene sequences in the packaging cell line utilized to produce the retroviral vector. Packaging cell lines in which the production of replication competent virus by recombination has been reduced or eliminated can be used to minimize the likelihood that a replication competent retrovirus will be produced. All retroviral vector supernatants used to infect cells are screened for replication competent virus by standard assays such as PCR and reverse transcriptase assays. Retroviral vectors allow for integration of a heterologous gene into a host cell genome, which allows for the gene to be passed to daughter cells following cell division.

Mammalian cell systems which utilize recombinant viruses or viral elements to direct expression can be engineered. For example, when using adenovirus expression vectors, the VEGF₁₄₅ protein coding sequence can be ligated to an adenovirus transcription/translation control complex, e.g., the late promoter and tripartite leader sequence. Alternatively, the vaccinia virus 7.5K promoter can be used (Mackett et al., Proc. Natl. Acad. Sci., USA 79:7415-7419, 1982; Mackett et al, J. Virol. 49:857-864, 1984; Panicali et al., Proc. Natl. Acad. Sci., USA 79:4927-4931, 1982). Particularly useful are bovine papilloma virus vectors, which can replicate as extrachromosomal elements (Sarver et al., Mol. Cell. Biol. 1:486, 1981). Shortly after entry of this DNA into mouse cells, the plasmid replicates to about 100 to 200 copies per cell. Transcription of the inserted cDNA yielding a high level of expression may result without integration of the plasmid into the host cell chromosome. These vectors can be used for stable expression by including a selectable marker in the plasmid, such as, for example, the neo gene. Alternatively, the retroviral genome can be modified for use as a vector capable of introducing and directing the expression of a VEGF₁₄₅ protein in the host cells (Cone and Mulligan, Proc. Natl. Acad. Sci., USA 81:6349-6353, 1984). High level expression can also be achieved using inducible promoters, including, but not limited to, the metallothionein IIA promoter and heat shock promoters.

Additional expression vectors containing regulatory elements from eukaryotic viruses such as retroviruses include the SV40 vectors (e.g., pSVT7 and pMT2), the bovine papilloma virus vectors (e.g., pBV-1MTHA) and vectors derived from Epstein Bar virus (e.g., pHEBO and p205). Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

It will be appreciated that the expression of the VEGF₁₄₅ polypeptide from the expression vector of the present invention can be specifically expressed endothelial cells by placing the polynucleotide sequence encoding this polypeptide under the transcriptional control of an endothelial specific promoter such as the promoter of Tie-2 (Lin, P. et al., 1988, Antiangiogenic gene therapy targeting the endothelium-specific receptor tyrosine kinase Tie2. Proc. Natl. Acad. Sci. USA. 95: 8829-8834).

Since VEGF₁₄₅ is the only secreted VEGF form that binds tightly to the extracellular matrix it will accumulate and concentrate on denuded surfaces where a layer of platelet and fibrin induces the deposition of extracellular matrix.

Thus, such a VEGF₁₄₅ polypeptide would also be beneficial in treating restenosis via the activation of the VEGFR-2 receptor on endothelial cells and the subsequent proliferation of endothelial cells at the denuded surface.

Thus, according to another aspect of the present invention there is provided a method of preventing and/or treating restenosis in an individual in need thereof comprising providing to the individual a VEGF₁₄₅ polypeptide exhibiting a VEGFR-2 binding activity and lacking a VEGFR-1 binding activity thereby preventing and/or treating restenosis in the individual in need thereof.

As used herein, the term restenosis refers to re-narrowing or blockage of the artery following angioplasty, stent placement or other PCI treatments.

It will be appreciated that in order to prevent and/or treat restenosis the VEGF₁₄₅ polypeptide of the present invention is provided to the artery and/or vein of the individual in need thereof.

Preferably, the VEGF₁₄₅ polypeptide of the present invention is delivered to the artery and/or vein from a stent which is placed therein. The stent can be coated with a releasable form of the VEGF₁₄₅ polypeptide variant of the present invention or an expression vector expressing same. Methods of coating stents with polypeptides or other therapeutic agents are well known in the arts (see for example, U.S. Pat. Appl. No. 20030207856 to Tremble, P. et al., and U.S. Pat. Appl. No. 20030215564 to Heller, P. F. et al.).

Briefly, in order to attach the VEGF₁₄₅ polypeptide variant of the present invention to a stent, a solution including a solvent, a polymer dissolved in the solvent and the VEGF₁₄₅ polypeptide variant which is dispersed within the solvent is prepared. It will be appreciated that for a successful stent coating the solvent and polymer used should be mutually compatible with therapeutic substance (e.g., the VEGF₁₄₅ polypeptide variant) in such a way that does not alter the therapeutic effect of the therapeutic substance. The solution is applied to the stent and the solvent is allowed to evaporate leaving a stent coating comprising the polymer(s) and the VEGF₁₄₅ polypeptide.

Typically, the solution can be applied to the stent by either spraying the solution onto the stent or immersing the stent in the solution depending on the viscosity and surface tension of the solution. However, for greatest uniformity and better control over the amount of coating material applied to the stent, spraying using a fine spray (e.g., such as that available from an airbrush) is preferable. In either case, multiple application steps are generally desirable to provide improved coating uniformity and improved control over the amount of VEGF₁₄₅ polypeptide to be applied to the stent. The total thickness of the polymeric coating will range from approximately 1 micron to about 20 microns or greater.

The polymer used by the present invention is biocompatible which minimizes irritation to the vessel wall during stent placement. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polyddioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Biostable polymers with a relatively low chronic tissue response such as polyurethanes, silicones, and polyesters could be used while other polymers could also be used if they can be dissolved and cured or polymerized on the stent; examples of the latter include polyolefins, polyisobutyleneand ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones, polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The ratio between the polymer and the VEGF₁₄₅ polypeptide variant will depend on the efficacy of the polymer in securing the polypeptide onto the stent and the rate at which the coating releases the VEGF₁₄₅ polypeptide to the tissue of the blood vessel. Large quantities of polymer material are required if the polymer has relatively poor efficacy in retaining the VEGF₁₄₅ polypeptide on the stent. Moreover, in cases where the therapeutic substance (e.g., the VEGF₁₄₅ polypeptide) is a highly soluble agent, large quantities of polymer are needed to provide an elution matrix that limits the elution of such an agent. A wide ratio of therapeutic substance-to-polymer could therefore be appropriate and could range from about 0.1% to 99% by weight of therapeutic substance-to-polymer.

It is expected that during the life of this patent many relevant VEGF₁₄₅ variants lacking a VEGFR-1 binding activity will be developed and the scope of the term VEGF₁₄₅ polypeptide variant is intended to include all such new variants a priori.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example I

### Construction of a VEGF₁₄₅ Variant that lacks VEGFR-1-Binding Ability

Since VEGF₁₄₅ is the only secreted VEGF form that binds tightly to the extracellular matrix it is expected to accumulate and concentrate on denuded surfaces of blood vessels without loss of bioactivity and thus assist in the process of re-endothelialization more efficiently than other VEGF forms (Poltorak, Z., et al., 1997. VEGF₁₄₅: a secreted VEGF form that binds to extracellular matrix. J. Biol. Chem. 272, 7151-7158). However, since VEGF₁₄₅ also binds and activates the VEGFR-1 receptor, which is expressed in macrophages, monocytes and SMC, its activation may result in the migration and proliferation of SMCs and finally lead to restenosis. In order to prevent the activation of the VEGFR-1 receptor by VEGF₁₄₅ a mutated variant of VEGF₁₄₅ was constructed as part of the present study.

Materials and Experimental Methods

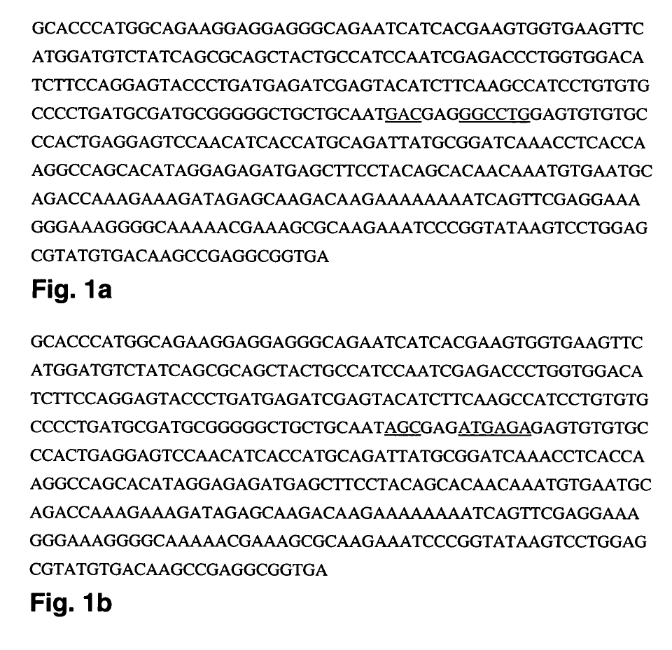

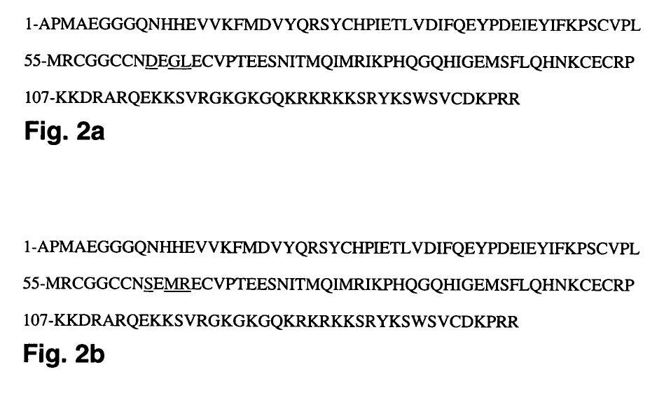

PCR directed mutagenesis—To generate the GAC→AGC, GGC→ATG and CTG→AGA site directed mutations (see FIGS. 1a-b, highlighted nucleotides) which correspond to the Asp63Ser, Gly65Met and Leu66Arg amino acid changes (FIGS. 2a-b, highlighted amino acids), respectively, the DNA coding the VEGF₁₄₅ protein form was subjected to two PCR reactions. The first PCR reaction used the V5 primer, which contains a BamHI site, and the pr60-3′ primer, which contains the AGC, ATG and AGA mutations (Table 1, hereinbelow). The second PCR reaction used the pr60-5′ primer and V3 primer, which contains a KpnI site (Table 1, hereinbelow). To assemble the mutated VEGF₁₄₅ cDNA the two PCR products were used a template for a third PCR reaction using the V5 and V3 PCR primers.

TABLE 1
PCR primers

| Primer | | |
|---|---|---|
| name | SEQ IDs | Primer 5′→3′ |
| V5 | SEQ ID NO: 5 | CGGGATCCGAAACCATGAACTTTCTGC |
| pr60-3′ | SEQ ID NO: 6 | TCCTCAGTGGGCACACACTCTCTCATCTCGCTATTGCAGCAGCCCC |
| pr60-5′ | SEQ ID NO: 7 | GAGTGTGTGCCCACTGAGGAGTCCAACATCA |
| V3 | SEQ ID NO: 8 | GGGGTACCCCTCACCGCCTCGGCTTGTC |

Table 1: The sequences of the PCR primers are provided from 5′→3′. Note that the underlined nucleotides in the V5 and V3 primers correspond to the BamHI and KpnI restriction sites, respectively and the bolded nucleotides in pr60-3′ correspond to the three introduced mutations: GAC→AGC, GGC→ATG and CTG→AGA.

Cloning of the mutated VEGF145 cDNA form—The assembled PCR product generated using the V5 and V3 PCR primers was digested using the BamHI and KpnI restriction enzymes (New England Biolabs, Beverly, Mass.) and was cloned in the KpnI and BanHI sites of the pcDNA3.1neo vector (Invitrogen Life Technologies, Frederick, Md., USA) vector.

DNA transfection and analysis of recombinant VEGF₁₄₅ protein—The cDNA coding the mutated VEGF₁₄₅ form was transiently transfected into Cos cells using the lipofectamine reagent (Gibco/Life Technologies, Rockville, Md.) and the conditioned medium was subjected to Western Blot analysis using anti-VEGF rabbit polyclonal antibodies as described elsewhere (Cohen, T., Gitay-Goren, H., Neufeld, G., and Levi, B.-Z. (1992) High levels of biologically active vascular endothelial growth factor (VEGF) are produced by the baculovirus expression system. Growth Factors. 7: 131-138).

Experimental Results

Construction of a mutated VEGF₁₄₅ cDNA form—To generate a VEGF₁₄₅ variant form that lacks the VEGFR-1 binding capacity, the cDNA coding the VEGF₁₄₅ form was subjected to the following site directed mutagenesis: Asp63Ser, Gly65Met and Leu66Arg as described hereinabove.

The mutated cDNA was cloned into the pcDNA3.1neo vector and was successfully transcribed and translated using a transient transfection assay.

Similar mutations (Asp63Ser, Gly65Met and Leu66Arg) which were introduced in the VEGF₁₆₅ form resulted in a VEGF₁₆₅ variant which binds to VEGFR-2 with normal affinity, however with 1900 fold less affinity to VEGFR-1 receptor (Li, B., et al., 2000. Receptor-selective variants of human vascular endothelial growth factor—Generation and characterization. J. Biol. Chem. 275: 29823-29828).

Thus, taking together the prior art results and the VEGF₁₄₅ variant of the present invention it is expected that the VEGF₁₄₅ variant of the present invention would bind to VEGFR-2 with normal affinity, however, would lack the ability to bind to the VEGFR-1 receptor.

Therefore, in order to prevent and treat restenosis the VEGF₁₄₅ variant of the present is administered into a tissue such as a denuded surface in a site of a coronary stent where it binds and activates the VEGFR-2 receptor on the endothelial cells and promotes re-endothelialization.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank accession numbers mentioned in this specification are herein incorporated in their entirety by reference into the specification, to the same extent as if each individual publication, patent or patent application or GenBank accession number was specifically and individually indicated to be incorporated herein by reference. In addition, citation or identification of any reference in this application shall not be construed as an admission that such reference is available as prior art to the present invention.

### REFERENCES

1. Welt, F. G. and Rogers, C. (2002) Inflammation and restenosis in the stent era. Arterioscler. Thromb. Vasc. Biol. 22, 1769-1776
2. Rutanen, J., Puhakka, H., and Yla-Herttuala, S. (2002) Post-intervention vessel remodeling. Gene Ther. 9, 1487-1491
3. Barleon, B., Sozzani, S., Zhou, D., Weich, H. A., Mantovani, A., and Marme, D. (1996) Migration of human monocytes in response to vascular endothelial growth factor (VEGF) is mediated via the VEGF receptor flt-1. Blood 87, 3336-3343
4. Wang, H. and Keiser, J. A. (1998) Vascular endothelial growth factor upregulates the expression of matrix metalloproteinases in vascular smooth muscle cells: role of flt-1. Circ. Res. 83, 832-840
5. Gluzman-Poltorak, Z., Cohen, T., Herzog, Y., and Neufeld, G. (2000) Neuropilin-2 is a receptor for the vascular endothelial growth factor (VEGF) forms VEGF-145 and VEGF-165 [corrected]. J. Biol. Chem. 275, 18040-18045
6. Poltorak, Z., Cohen, T., Sivan, R., Kandelis, Y., Spira, G., Vlodavsky, I., Keshet, E., and Neufeld, G. (1997) VEGF145: a secreted VEGF form that binds to extracellular matrix. J. Biol. Chem. 272, 7151-7158
7. Zhao, Q., Egashira, K., Inoue, S., Usui, M., Kitamoto, S., Ni, W., Ishibashi, M., Hiasa, K. K., Ichiki, T., Shibuya, M., and Takeshita, A. (2002) Vascular endothelial growth factor is necessary in the development of arteriosclerosis by recruiting/activating monocytes in a rat model of long-term inhibition of nitric oxide synthesis. Circulation 105, 1110-1115
8. Kendall, R. L., Wang, G., and Thomas, K. A. (1996) Identification of a natural soluble form of the vascular endothelial growth factor receptor, FLT-1, and its heterodimerization with KDR. Biochem Biophys. Res. Commun. 226, 324-328
9. Gille, H., Kowalski, J., Li, B., LeCouter, J., Moffat, B., Zioncheck, T. F., Pelletier, N., and Ferrara, N. (2001) Analysis of biological effects and signaling properties of Flt-1 and KDR: A reassessment using novel highly receptor-specific VEGF mutants. J. Biol. Chem. 276, 3222-3230
10. Siemeister, G., Schirner, M., Reusch, P., Barleon, B., Marme, D., and Martiny-Baron, G. (1998) An antagonistic vascular endothelial growth factor (VEGF) variant inhibits VEGF-stimulated receptor autophosphorylation and proliferation of human endothelial cells. Proc. Natl. Acad. Sci. USA 95,4625-4629
11. Li, B., Fuh, G., Meng, G., Xin, X. H., Gerritsen, M. E., Cunningham, B., and De Vos, A. M. (2000) Receptor-selective variants of human vascular endothelial growth factor—Generation and characterization. J. Biol. Chem 275, 29823-29828
12. Keyt, B. A., Nguyen, H. V., Berleau, L. T., Duarte, C. M., Park, J., Chen, H., and Ferrara, N. (1996) Identification of vascular endothelial growth factor determinants for binding KDR and FLT-1 receptors—Generation of receptor-selective VEGF variants by site—directed mutagenesis. J. Biol Chem 271, 5638-5646
13. Lange, T., Gutman-Raviv, N., Baruch, L., Machluf, M., and Neufeld, G. (2002) VEGF₁₆₂: A new heparin binding VEGF splice form that is expressed in transformed human cells. Submitted
14. Cohen, T., Gitay-Goren, H., Neufeld, G., and Levi, B.-Z. (1992) High levels of biologically active vascular endothelial growth factor (VEGF) are produced by the baculovirus expression system. Growth Factors. 7, 131-138
15. Peretz, D., Gitay-Goren, H., Safran, M., Kimmel, N., Gospodarowicz, D., and Neufeld, G. (1992) Glycosylation of vascular endothelial growth factor is not required for its mitogenic activity. Biochem. Biophys. Res. Commun. 182, 1340-1347
16. Gluzman-Poltorak, Z., Cohen, T., Shibuya, M., and Neufeld, G. (2001) Vascular endothelial growth factor receptor-1 and neuropilin-2 form complexes. J. Biol. Chem. 276, 18688-18694
17. Soker, S., Takashima, S., Miao, H. Q., Neufeld, G., and Klagsbrun, M. (1998) Neuropilin-1 is expressed by endothelial and tumor cells as an isoform specific receptor for vascular endothelial growth factor. Cell 92, 735-745
18. Gluzman-Poltorak, Z., Cohen, T., Herzog, Y., and Neufeld, G. (2000) Neuropilin-2 and Neuropilin-1 are receptors for 165-amino acid long form of vascular endothelial growth factor (VEGF) and of placenta growth factor-2, but only neuropilin-2 functions as a receptor for the 145 amino acid form of VEGF. J. Biol Chem 275, 18040-18045
19. Savani, R. C., Wang, C., Yang, B., Zhang, S., Kinsella, M. G., Wight, T. N., Stern, R., Nance, D. M., and Turley, E. A. (1995) Migration of bovine aortic smooth muscle cells after wounding injury. The role of hyaluronan and RHAMM. J. Clin. Invest 95, 1158-1168
20. Lemire, J. M., Merrilees, M. J., Braun, K. R., and Wight, T. N. (2002) Overexpression of the V3 variant of versican alters arterial smooth muscle cell adhesion, migration, and proliferation in vitro. J. Cell Physiol 190, 38-45
21. Itano, N., Atsumi, F., Sawai, T., Yamada, Y., Miyaishi, O., Senga, T., Hamaguchi, M., and Kimata, K. (2002) Abnormal accumulation of hyaluronan matrix diminishes contact inhibition of cell growth and promotes cell migration. Proc. Natl. Acad. Sci U.S.A. 99, 3609-3614

What is claimed is:

1. An isolated VEGF₁₄₅ polypeptide devoid of a VEGFR-1 binding activity.

2. The isolated VEGF₁₄₅ polypeptide of claim 1, wherein said polypeptide is set forth by SEQ ID NO:4.

3. The isolated VEGF₁₄₅ polypeptide of claim 1, wherein said polypeptide is capable of binding to VEGFR-2.

4. An isolated polynucleotide comprising a nucleic acid sequence encoding a VEGF₁₄₅ polypeptide devoid of a VEGFR-1 binding activity.

5. The isolated polynucleotide of claim 4, wherein said polynucleotide is set forth by SEQ ID NO:2.

6. The isolated polynucleotide of claim 4, wherein said polynucleotide is at least 90% homologous to the polynucleotide sequence set forth by SEQ ID NO:1 as determined using the BlastN software of the National Center of Biotechnology Information (NCBI) using default parameters.

7. The isolated polynucleotide of claim 4, wherein said VEGF₁₄₅ polypeptide exhibits a VEGFR-2 binding activity.

8. A nucleic acid construct comprising the isolated polynucleotide of claim 4.

9. The nucleic acid construct of claim 8, further comprising a promoter for directing expression of the isolated polynucleotide in mammalian cells.

10. The nucleic acid construct of claim 9, wherein said promoter is an endothelial cell specific promoter.

11. A method of promoting re-endothelialization in a tissue of an individual comprising providing to the tissue of the individual a VEGF₁₄₅ polypeptide exhibiting a VEGFR-2 binding activity and lacking a VEGFR-1 binding activity thereby promoting re-endothelialization in the tissue of the individual.

12. The method of claim 11, wherein said tissue is selected from the group consisting of an artery and a vein.

13. The method of claim 11, wherein said VEGF₁₄₅ polypeptide is set forth by SEQ ID NO:4.

14. The method of claim 11, wherein said providing is effected by administering said VEGF₁₄₅ polypeptide into the tissue of the individual.

15. The method of claim 11, wherein said providing is effected by expressing a polynucleotide encoding said VEGF₁₄₅ polypeptide in the tissue of the individual.

16. The method of claim 15, wherein said polynucleotide is set forth by SEQ ID NO:2.

17. The method of claim 15, wherein said polynucleotide at least 90% homologous to the polynucleotide sequence set forth in SEQ ID NO:1 as determined using the BlastN software of the National Center of Biotechnology Information (NCBI) using default parameters.

18. A method of preventing and/or treating restenosis in an individual in need thereof comprising providing to the individual a VEGF₁₄₅ polypeptide exhibiting a VEGFR-2 binding activity and lacking a VEGFR-1 binding activity thereby preventing and/or treating restenosis in the individual in need thereof.

19. The method of claim 18, wherein said VEGF₁₄₅ polypeptide is set forth by SEQ ID NO:4.

20. The method of claim 18, wherein said providing is effected by administering said VEGF₁₄₅ polypeptide into the individual.

21. The method of claim 18, wherein said providing is effected by expressing a polynucleotide encoding said VEGF₁₄₅ polypeptide in an artery and/or a vein of the individual.

22. The method of claim 21, wherein said polynucleotide is set forth by SEQ ID NO:2.

23. The method of claim 21, wherein said polynucleotide at least 90% homologous to the polynucleotide sequence set forth in SEQ ID NO:1 as determined using the BlastN software of the National Center of Biotechnology Information (NCBI) using default parameters.
